(19) United States
(12) Patent Application Publication
(10) Pub. No.: US 2005/0131324 A1
(43) Pub. Date: Jun. 16, 2005

(54) Boot for treatment of plantar fasciitis

(76) Inventor: Gary R. Bledsoe, Mansfield, TX (US)

Correspondence Address: LOCKE LIDDELL & SAPP LLP;ATTN: SUE COTT, 2200 ROSS AVENUE, SUITE 2200, DALLAS, TX, 75201-6776, US

(21) Appl. No.: 10/737,396

(22) Filed: Dec. 15, 2003

(51) Int. Cl. A61F 5/00

(52) U.S. Cl. 602/23

(57) ABSTRACT

A boot for use in the treatment of plantar fasciitis. The boot places the foot in the desired amount of dorsiflexion, preferably from 5° to 20°, which stretches the Achilles tendon and is believed to prevent the shortening of the plantar fascia. The boot also provides protection for the foot while walking or engaging in other load bearing activities, to minimize further damage to and/or inflammation of the plantar fascia. The boot has a sole that is shaped to allow as close to a natural walking gait as possible while maintaining the foot in the desired degree of dorsiflexion. Preferably, the amount of dorsiflexion of the foot is controlled through the addition wedges on the base of the boot.

### BACKGROUND OF THE INVENTION

1. Field of the Invention

This invention relates generally to a boot that can be used for the treatment of plantar fasciitis and, more particularly, to a boot that is worn both day and night during the treatment period.

2. Description of Related Art

Plantar Fasciitis is a medical condition that results in pain in one or both feet, generally localized in the heels. The pain associated with Plantar Fasciitis, ranging from a dull intermittent pain to a sharp persistent pain, may continue for months or even years and in some cases the pain may never completely disappear. It is often most severe immediately after getting out of bed in the morning or after other periods of inactivity. The condition is more common in people who spend a lot of time on their feet, walk or run on hard surfaces, have put on extra pounds, have tight Achilles tendons, or have had a sudden increase in activity after a period of inactivity.

The cause of Plantar Fasciitis is unknown but is believed to be damage to and inflammation of the plantar fascia, especially where it connects to the heel. The planter fascia is a highly elastic collagen-based connective tissue that runs between the heel and the toes. It helps to support the arch of the foot as well as helps check the motion of the various joints in the ankle and foot. When these joints are stressed, the plantar fascia may become inflamed. In addition, both the plantar fascia and the Achilles tendon tend to shorten up to some extent at night and during other periods of inactivity. This shortening results in increased amounts of stress being placed upon the connection between the plantar fascia and the heel, upon the resumption of movement. It is the additional stress applied to either the joints in the foot or the connection between the plantar fascia and the heel that is believed to result in the inflammation and pain associated with Plantar Fasciitis.

For mild cases, a number of simple remedies have been used with some degree of success. For example, stretching exercises have been used to loosen the Achilles tendon and the plantar fascia so as to place less tension on the connection between the fascia and the heel. Cutting back on pounding exercises, such as jogging on hard surfaces may also help. Alternatively, anti-inflammatory drugs such as naproxen or ibuprofen may also help in some cases. Orthotic inserts for the shoe and walking boots can also be used to minimize stresses applied to the foot when standing or walking and help protect the plantar fascia from further damage and inflammation, thereby allowing the plantar fascia to properly heal.

Wearing a splint at night may also provide some relief. The splint holds the plantar fascia tensioned during the night. This is accomplished either by applying a compressive force directly against the plantar fascia or by placing the foot in a slight dorsiflexion position to tension the plantar fascia by stretching it slightly. The tension is believed to help ease inflammation or decrease the stress applied to the plantar fascia in the morning when activity resumes, thereby reducing the pain associated with plantar fasciitis. However, it is not intended that these splint devices be used when weight is being placed on the foot, other than brief occasions such as walking to the bathroom during the night. As such, these splints are not designed to allow a user to have a near natural walking gait while wearing the brace or for protecting the foot while standing or walking. Therefore, night splints are not useful for providing protection or relieving pain during the day because the wearer cannot carry out normal ambulatory movements. Daily activities allow additional stress and micro tears to occur in the plantar fascia due to normal stresses that are applied upon already inflamed and injured tissues. This aggravates the plantar fasciitis and minimizes the effectiveness of the night splint, especially for more severe cases of plantar fasciitis.

For the most severe cases, surgery may be necessary to loosen the plantar fascia by snipping a part of it. However, this requires an extended recovery period during which pain is often more severe. Therefore, while a number of treatment options are available to treat plantar fasciitis, there is still a need for a device that has the ability to treat plantar fasciitis across a wide range of severity while allowing the patient to continue to engage in their normal daily activities.

### SUMMARY OF THE INVENTION

A boot for use in the treatment of plantar fasciitis. The boot places the foot in the desired amount of dorsiflexion, preferably from 5° to 20°, which stretches the Achilles tendon and is believed to prevent the shortening of the plantar fascia. The boot also provides protection for the foot while walking or engaging in other load bearing activities, to minimize further damage to and/or inflammation of the plantar fascia. The boot has a sole that is shaped to allow as close to a natural walking gait as possible while maintaining the foot in the desired degree of dorsiflexion. Preferably, the amount of dorsiflexion of the foot is controlled through the addition of a number of wedges on the base of the boot.

### BRIEF DESCRIPTION OF THE DRAWINGS

The apparatus of the invention is further described and explained in relation to the following figures wherein:

FIG. 1 is a perspective view of a preferred embodiment of the boot of the current invention using three wedges and fitted onto a patient's foot;

FIG. 2 is a perspective view of the boot shown in FIG. 1 with all of the straps, pads and cuffs removed;

FIG. 3 is a side elevation view of the base of the boot shown in FIG. 1;

FIG. 4 is a is a plan view of the cuff of the boot show in FIG. 1; and

FIG. 5 is a side elevation view of a wedge.

### DETAILED DESCRIPTION OF THE PREFERRED EMBODIMENT

The current invention provides a novel device for the treatment of plantar fasciitis. In a preferred embodiment, this is accomplished by maintaining the plantar fascia is a position where it can properly heal and protecting it from further damage from walking or bearing weight upon the foot. It has been found that approximately 70% of patients with plantar fasciitis have tightened Achilles tendons. Stretching provides temporary relief of minor to moderate cases of plantar fasciitis, however, the Achilles tendon will begin to shorten back up again once it is relaxed. In the preferred embodiment, the boot positions the foot in a degree of dorsiflexion. This continuously maintains the Achilles tendon in a moderately stretched state. Preferably, this stretched state is slowly increased over time, e.g. a couple weeks, to further stretch the Achilles tendon and provide further relief as directed by a physician. It is believed that maintaining the foot with a certain amount of dorsiflexion also provides tension on the plantar fascia itself. This tension is believed to help the plantar fascia remodel itself as it heals so that it is less likely to be re-injured from normal activities.

The boot of the preferred embodiment can also be worn while standing or walking. This allows the boot to continue to maintain the Achilles tendon in a stretched position and provide tension on the plantar fascia to allow it to properly heal. It also provides protection of the plantar fascia during walking and standing activities that normally produce additional strain upon the foot and plantar fascia and could cause additional damage and inflammation. The protection is provided both by cushioning foot 58 and by transmitting forces through base 22 and up upright arms 24 to leg 60 instead of allowing the forces to pass through foot 58. As such, it is contemplated that boot 20 will be worn all day and night during the treatment period. For use at night, a spandex/lycra cover (not shown) can be used to cover base 22 so that it does not soil the bed linens. Boot 20 can be removed for range of motion and strengthening exercises or they can be carried out while wearing the boot, as recommended by the treating physician.

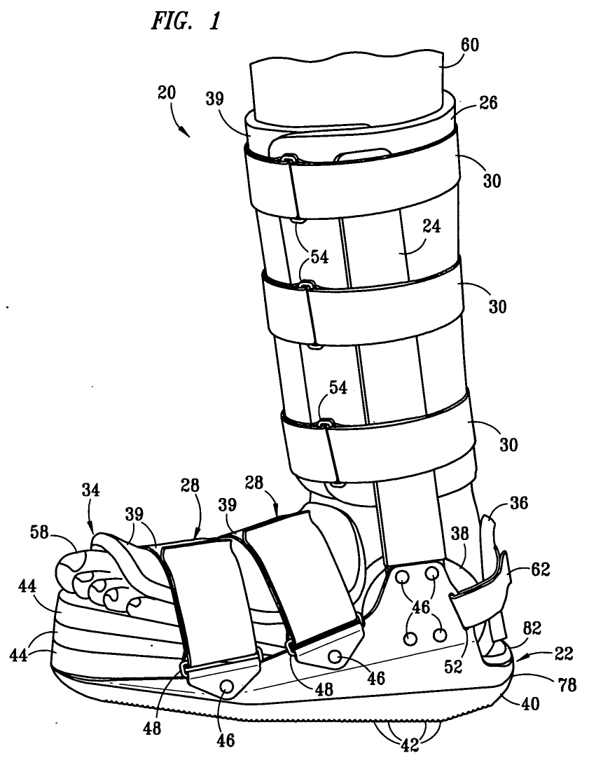

Referring to FIG. 1, boot 20 is generally made up of base 22, upright arms 24, cuff 26, and wedges 44. In addition, foot straps 28 and leg straps 30 are provided to help hold the foot and leg in boot 20. Calf cuff 26, footpad 34, heel pad 36 and ankle pads 38 are made of a breathable foam in order to keep the skin cool and comfortable while wearing boot 20. Straps 28 and 30 as well as calf cuff 26, footpad 34, heel pad 36 and ankle pads 38 have pile surface 39 on at least one side that allows hook fasteners 28 to be secured to them to form a hook and loop fastening system, e.g., Velcro. Pile surface 39 may be an integral part of the respective strap or pad or may be a separate layer that is secured to the strap or pad through means known to those of skill in the art, e.g. heat fusion, adhesive, stitching, etc. In addition, those skilled in the art will recognize that other type of fasteners, e.g. snaps, buckles, or other methods to secure the straps, pads and cuff of boot 20 together may be used without departing from the current invention. Hook and loop fasteners are preferred because they are sufficiently sturdy, easy to adjust, and simple to construct and use.

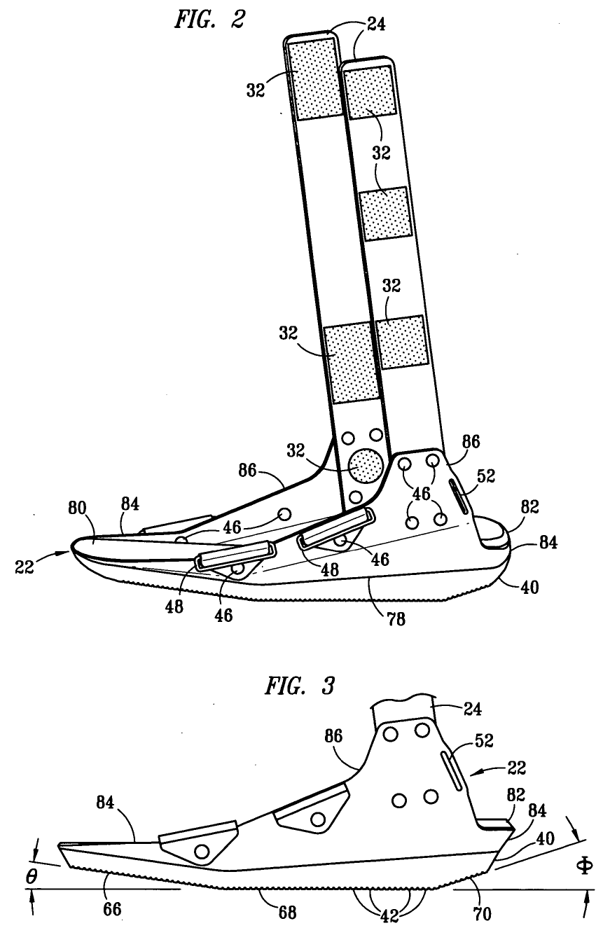

Referring to FIG. 2, base 22 is made up of shell 78, sole 40 and insole 80. Shell 78 is preferably made of an aluminum alloy in order to impart form and rigidity to base 22. Shell 78 has lip 84 running around the edge of shell 78. Lip 84 widens into sides 86 where shell 78 connects to upright arms 24. D-Rings 48 are attached by rivets 46 to either side of shell 78. Also slots 52 are located in both sides 86 of shell 82 to accommodate heel strap 62. Insole 80 is dense foam cushion material that is attached to the interior of shell 78. Insole 80 provides a cushioned surface for foot 58 to rest on when boot 20 is worn. However, insole 80 is firm enough to prevent foot 58 from moving significantly out of the desired degree of dorsiflexion. Pad 82 is located at the rear of shell 78 on lip 84 and also provides padding to protect foot 58 from contacting shell 78. Sole 40 is attached to the bottom of shell 78.

Referring to FIG. 3, sole 40 is shaped to allow the patient to have a near normal gait while walking in boot 20. Sole 40 is preferably made of microcellular polyurethane so that it is light and very durable. Sole 40 also has treads 42 on the bottom in order to help provide traction and prevent slips. Sole 40 is made up of three generally flat surfaces, front surface 66, center surface 68 and rear surface 70. Surfaces 66, 68, and 70 are connected to each other with arcuate areas to allow boot 20 to smoothly rock from one surface to the next. Rear surface 70 absorbs the shock of boot 20 striking the ground and thereby protects the joints of foot. 58 from these forces. Center surface 68 provides a surface suitable for supporting the wearer's weight without placing undue stresses on either the plantar fascia or the joints in foot 58, when the wearer is in a standing weight bearing position. Front surface 66 is angled to allow the wearer to easily push off the ground when taking a step.

During walking, boot 20 generally first strikes the ground on rear surface 70, which is at an angle Φ to help cushion the impact with the ground and then rocks through center surface 68 to front surface 66, which is at an angle 0 to aid in pushing the boot off the ground to take the next step. In this manner, the shape of sole 40 allows the wearer to have as close to a normal walking gait as possible while maintaining foot 58 at the desired degree of dorsiflexion. Thus the shape of sole 40 minimizes the decrease in mobility from wearing a walking boot. This decreases the inconvenience of the boot to the wearer and increases the chance that boot 20 will be worn for the entire time prescribed by the treating physician.

Referring back to FIG. 2, upright arms 24 are flat strips of metal on either side of base 22 that extend upward in a generally vertical direction. Rivets 46 connect the bottom of upright arms 24 to shell 78 at sides 86. Upright arms 24 preferably have a slight outward flare to better conform to the shape of the leg 60. Upright arms 24 are preferably composed of an aluminum alloy with a fabric cover sewn over it. Each upright arm 24 contains a number of hook fasteners 32 on both the outside and the inside of upright arms 24. Hook fasteners 32 can attach to pile surface 39 on the outside of cuff 26 as well as leg straps 30. Also, at the bottom of the interior side of each upright arm 24 are hook fasteners 32. Hook fasteners 32 attach to pile surface (not shown) on ankle pads 38 in order to secure ankle pads 38 to the inside of upright arms 24. As can be seen more clearly in FIG. 1, ankle pads 38 serve to cushion foot 58 and protect it from rubbing against upright arms 24 or rivets 46.

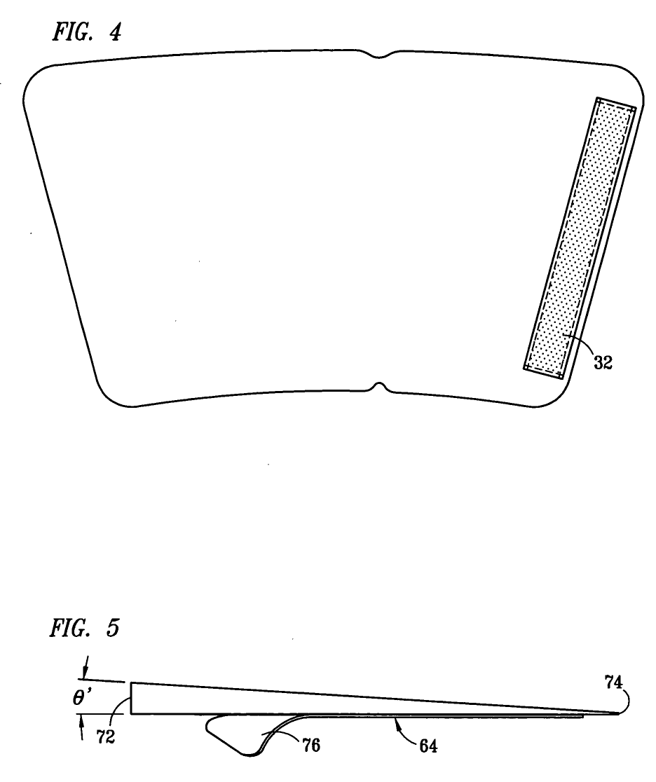

Referring to FIG. 4, cuff 26 is made of flexible breathable foam that can be wrapped around leg 60. Hook fastener 32 runs along one vertical edge of cuff 26 and allows one end of cuff 26 to be secured to pile surface 39 on the outside of the other end of cuff 26 after it has been wrapped around leg 60. Preferably the length of cuff 26 is adjusted by trimming cuff 26 along the edge opposite from fastener 32 in order to provide approximately 2-3 inches of overlap when cuff 26 is wrapped around leg 60.

As can be seen in FIG. 1, each wedge 44 is shaped to fit on top of base with front edge 72 lined up with the front edge of base 22. Referring to FIG. 5, each wedge 44 has a thick front edge 72 that angles down to a thin rear edge 74. The angle of wedge 44 is such that angle θ′ is 5°. When positioned on base 22, rear edge 74 of wedge 44 does not cover all of base 22 but rather terminates short of the rear of base 22. Wedges 44 are made of dense foam. On the bottom of each wedge 44 is adhesive strip 64. Adhesive strip 64 preferably contains a paper cover 76 that peels off when ready for use. Adhesive strip 64 is used to secure each wedge 44 to either wedge 44 below it or base 22, in order to prevent wedges 44 from slipping, especially while the wearer is walking in boot 20. There are preferably three wedges 44 provided with boot 20 so that the degree of dorsiflexion of foot 58 can be controlled in 5° increments between 5° and 20°.

Foot straps 28 are secured to D-rings 48 on one side of base 22. Foot straps 28 have one side that has a loose pile surface for attachment to two-sided hook fasteners. Two sided hook fasteners are the same as hook fasteners 32 except they have hook fasteners on both surfaces so that they can secure two pile surfaces 39 together. Hook fasteners 32 are secured on the other surface of foot straps 28 in order to locate footpad 34 on top of foot 60. Foot straps 28 are passed through D-rings 48 on the other side of base 22 and secured to themselves using two-sided hook fasteners. In this way foot straps 28 can be secured snugly around foot 60 and hold it in place on base 22 with footpad 34 providing cushioning between foot straps 28 and foot 60. Likewise heel strap 62 is passed through slot 52 and secured to itself using a two-sided hook fastener. Hook fastener 32 is located on the inner surface of heel strap 62 to secure heel pad 36 in the desired location.

Leg straps 30 are formed of a thick breathable foam material with piled surface 39 on the outer surface for hook fasteners 32 to attach. Hook fasteners 32 are disposed at one end of each leg strap 30. At the other end of each leg strap 30 is ring 54. The end of leg strap 30 that has hook fastener 32 passes through ring 54 and doubles back so hook fastener 32 can attach to a portion of leg strap 30. Each leg strap 30 is also secured to cuff 26 by two-sided hook fasteners that are placed on the back of cuff 26 after it is wrapped around leg 60. Hook fasteners 32 are located on each upright arm 24 to secure each leg strap 30 to upright arms 24, as seen in FIG. 7. In this manner, leg straps 30 assist in securing cuff 26 to upright arms 24, thereby maintaining leg 60 in alignment with upright arms 24 and helping transmitting forces from the bottom of boots 20 to leg 60 so they avoid adding stress to the joints of foot 58. This helps ensure that foot 58 remains in the desired degree of dorsiflexion and is protected from forces generated while standing or walking.

Boot 20 may be secured to foot 58 and leg 60 in the following manner. First, a dry sock (not shown) is preferably placed on foot 58. Next, cuff 26 is wrapped snugly around leg 60 with the bottom just above the anklebones of leg 60. Cuff 36 is secured to the outside of itself through the use of hook fastener 32. Then foot 58 is placed directly on base 22 without any wedges 44 between the two. Foot 58 should be facing forward and leg 60 is lined up with upright arms 24. Hook fasteners 32 on the inside of upright arms 24 are attached to the outside of cuff 26, by pressing upright arms 24 together, to secure cuff 26 to both upright arms 24.

Heel pad 36 is then positioned on heel strap 62 using a two-sided hook fasteners and heel strap 62 is tightened snugly against the back of the heel by attaching to itself using two-sided hook fastener 50. Footpad 34 is then similarly located on foot straps 28 and foot straps 28 are secured back onto themselves also using two-sided hook fasteners to snugly hold foot 58 on base 22 and minimized movement of foot 58. Finally, leg straps 30 are wrapped around uprights 24 and cuff 26, and secured to hook fasteners 32 on upright arms 24 and two-sided hook fasteners placed on the back of cuff 26. Leg straps 30 are then passed through rings 54 and attached back onto themselves using two-sided hook fasteners.

Without the use of wedges 44, boot 20 preferably places the foot in 5° of dorsiflexion. After a period of time specified by the treating doctor, such as a week, one wedge 44 is added to the top of base 22 with the thick end of wedge 44 lined up with the front end of base 22 and secured to base 22 through the use of adhesive strip 64 on the bottom of wedge 44. Boot 20 is then refitted onto foot 58 and leg 60 as described above. The use of one wedge 44 increases the dorsiflexion of the foot by an additional 5° for a total of 10° of dorsiflexion. After successive time periods, additional wedges 44 are added one on top of the other and secured through the use of adhesive strips 64 on the bottom of each wedge 44. The degree of dorsiflexion of foot 58 is increased 5° for every additional wedge 44 that is used. In this way the desired degree of dorsiflexion can be controlled and slowly increased over a period of time. In addition, boots 20 serve to absorb some of the forces of impact of foot 58 on the ground and instead transmits such forces up upright arms 24 to leg 60, thereby protecting the plantar fascia and joints in foot 58 from receiving stresses that could cause further injury, inflame the plantar fascia, or delay healing.

The above descriptions of certain embodiments are made for the purposes of illustration only and are not intended to be limiting in any manner. Other alterations and modifications of the preferred embodiment will become apparent to those of ordinary skill in the art upon reading this disclosure, and it is intended that the scope of the invention disclosed herein be limited only by the broadest interpretation of the appended claims to which the inventor is legally entitled.

1. A device for treating plantar fasciitis comprising:
a boot comprising a shell, a sole on bottom of said shell and a footplate on top of said shell;
at least one upright connected to said shell;
a cuff to secure said at least one upright to a lower portion of a leg;
at least one strap to secure said boot to said foot;
wherein said sole has a bottom shaped to allow a near normal walking gait; and
wherein said footplate places said foot in an amount of dorsiflexion.

2. The device of claim 1 wherein there are two said at least one upright.

3. The device of claim 2 wherein said cuff is secured to said uprights by hook and eye closures.

4. The device of claim 2 further comprising at least one leg strap to further secure said calf cuff to said uprights.

5. The device of claim 1 wherein said footplate places said foot in 5° of dorsiflexion.

6. The device of claim 1 further comprising at least one wedge that can be placed on said footplate to increase said amount of dorsiflexion.

7. The device of claim 6 wherein each of said at least one wedge comprises an adhesive strip to secure said at least one wedge on said boot.

8. The device of claim 6 wherein each of said at least one wedge places said foot in an additional 5° of dorsiflexion.

9. The device of claim 1 wherein said at least one strap further comprises a foot strap and a heel strap.

10. The device of claim 9 further comprising pads attached to said at least one strap.

11. The device of claim 1 wherein said sole comprises a rear angle portion, a center portion, and a front angle portion.

12. The device of claim 11 wherein said portions of said sole are joined by arcuate areas.

13. The device of claim 1 wherein said cuff is made of breathable foam.

14. The device of claim 1 wherein said shell and said at least one upright are made of metal.

15. The device of claim 14 wherein said metal is aluminum alloy.

16. The device of claim 1 wherein said sole is made of microcellular polyurethane.

17. The device of claim 1 further comprising a cover for said sole portion of said boot.

18. A device for treating plantar fasciitis comprising:
a boot comprising a shell, a sole on bottom of said shell and a footplate on top of said shell;
at least one upright connected to said shell;
a cuff to secure said at least one upright to a lower portion of a leg;
at least one strap to secure said boot to said foot;
at least one wedge that can be placed on said footplate to increase said amount of dorsiflexion;
wherein said footplate places said foot in an amount of dorsiflexion; and
wherein said sole has a bottom shaped to allow a near normal walking gait.

19. The device of claim 18 wherein there are two said at least one upright.

20. The device of claim 18 wherein said cuff is secured to said at least one upright by hook and eye closures.

21. The device of claim 18 further comprising a number of leg straps to further secure said calf cuff to said at least one upright.

22. The device of claim 18 wherein said footplate places said foot in 5° of dorsiflexion.

23. The device of claim 18 wherein each of said at least one wedge places said foot in an additional 5° of dorsiflexion.

24. The device of claim 18 wherein each said at least one wedge comprises an adhesive strip to secure said at least one wedge on said boot.

25. The device of claim 18 wherein said at least one strap comprises a foot strap and a heel strap.

26. The device of claim 18 wherein and said calf cuff is made of breathable foam.

27. The device of claim 18 wherein said shell and said at least one upright are aluminum alloy.

28. The device of claim 18 wherein said sole is made of microcellular polyurethane.

29. A device for treating plantar fasciitis comprising:
a boot comprising a shell, a sole on bottom of said shell and a footplate on top of said shell;
two uprights connected to said shell;
ankle pads attached to each said two uprights.
a cuff to secure said two uprights to a lower portion of a leg;
at least one leg strap securing said cuff to said two uprights;
at least one wedge capable of placement on said footplate to increase said amount of dorsiflexion an additional 5°;
an adhesive strip on bottom of each said at least one wedge;
a foot strap and a heel strap attached to said shell;
pads on said straps;
wherein said footplate places said foot in 5° of dorsiflexion; and
wherein said sole has a bottom shaped to allow a near normal walking gait.

30. A method of treating plantar fasciitis comprising:
placing a foot into a boot, comprising a sole shaped to protect said foot while allowing a near normal walking gait and a footplate that places said foot in an amount of dorsiflexion with respect to a leg;
adjusting straps to secure said foot in said boot;
repeatedly adjusting said footplate after successive periods of time to increase dorsiflexion of said foot by a certain amount, until a maximum amount of dorsiflexion is achieved.

31. The method of claim 30 wherein said certain amount of dorsiflexion is increased by adding a wedge onto said footplate.

32. The method of claim 30 wherein said maximum amount of dorsiflexion is 20°.

33. The method of claim 30 wherein said amount of dorsiflexion is 5°.

34. The method of claim 30 wherein said boot is worn both during the day and at night.

35. The method of claim 34 further comprising placing a cover over said boot prior to going to sleep.

36. The method of claim 30 further comprising the step of protecting said foot during load bearing activities.
